(19) United States
(12) Patent Application Publication
(10) Pub. No.: US 2007/0179602 A1
(43) Pub. Date: Aug. 2, 2007

(54) Method and Devices For Cardiac Valve Annulus Expansion

(75) Inventor: John T. M. Wright, Denver, CO (US)

Correspondence Address: SWANSON & BRATSCHUN L.L.C., 1745 SHEA CENTER DRIVE, SUITE 330, HIGHLANDS RANCH, CO, 80129, US

(73) Assignee: GENESEE BIOMEDICAL, INC., Denver, CO (US)

(21) Appl. No.: 11/627,840

(22) Filed: Jan. 26, 2007

Related U.S. Application Data
(60) Provisional application No. 60/763,033, filed on Jan. 27, 2006.
(60) Provisional application No. 60/788,847, filed on Apr. 3, 2006.

(51) Int. Cl. A61F 2/24

(52) U.S. Cl. 623/2.11; 623/2.36; 606/198

(57) ABSTRACT

A method and apparatus is disclosed that allows a cardiac surgeon to temporarily and controllably dilate the aortic root using a thin walled spiral cylinder that is irreversibly dilated by an expansion apparatus. Following removal of the reversible expansive apparatus the irreversible dilation member temporarily remains in the expanded aortic root during aortic valve prosthesis placement with the irreversible dilation member at the level of the aortic annulus. Implantation sutures that had been previously placed are tied following the removal of the irreversible dilation member. The apparatus and method may be used to place a sub aortic stent for the treatment of sub aortic stenosis.

### RELATED APPLICATIONS

This application claims priority from U.S. Provisional Patent Application Ser. No. 60/763,033, filed Jan. 27, 2006 and 60/788,847, filed Apr. 3, 2006, entitled “Method and Devices For Cardiac Valve Annulus Expansion,” which are each hereby incorporated by reference.

### TECHNICAL FIELD

The present invention is directed toward a cardiac valve annulus expansion device and a method of using a cardiac valve annulus expansion device.

### BACKGROUND OF THE INVENTION

A normal human aortic valve is a tri-leaflet valve with three nearly equally sized cusps. The aortic valve lies in the outflow tract of the left ventricle and at the base of the aorta. Aortic valve disease is a significant disease, and if untreated, progresses and eventually often leads to premature death. The three main types of aortic valve disease are: aortic stenosis; aortic regurgitation; and bacterial endocarditis. The latter can occur at almost any age. Pure aortic regurgitation is unusual, but aortic stenosis is relatively common. The typical adult patient presenting with aortic stenosis is a male patient about 60 years old with a congenital valve defect in which one of the three leaflets is either rudimentary (i.e. unusually small) or absent. This type of valve defect is usually referred to as a bicuspid valve.

Infected valves almost universally have to be surgically replaced. Very, occasionally a regurgitant valve may be successfully repaired (at least in the short term), but in almost all patients presenting for surgery the stenotic valve is replaced by mechanical, bioprosthetic, or homograft valve.

Aortic stenosis is defined by a significantly higher than normal pressure gradient across the aortic valve. The increased gradient raises the left ventricular ejection pressure and hence energy requirements of the heart. The blood supply to the heart including the left ventricle is from the coronary arteries originating distal to the aortic valve, and coronary perfusion of the coronary arteries occurs in ventricular diastolie when the left ventricular myocardium is relaxed. During ventricular systolie, the left ventricular wall stress is elevated significantly above coronary perfusion pressure. Hence coronary perfusion is not possible during ventricular systolie. In patients with aortic stenosis whilst the metabolic requirements of the heart increase significantly, the energy supply to the myocardium does not, because diastolic blood pressure remains near normal. Hence the heart suffers an energy deficit which is uncompensated. This is usually not apparent to the patient. Consequently, aortic stenosis may be an asymptomatic disease, symptoms only appearing in end-stage disease. Studies have demonstrated that 85% of patients with surgically untreated aortic stenosis die within 5 years after the onset of symptoms. It follows that an important characteristic of a replacement aortic valve is minimal aortic pressure gradient, especially in symptomatic patients. This criterion dictates that a prosthesis with a sufficiently large internal orifice diameter be implanted. It has been shown that the pressure gradient across a prosthetic heart valve is inversely proportional to the fourth power of the internal orifice diameter of the prosthesis. It follows that the external diameter of the valve (including that of the sewing cushion) is also extremely important, as the orifice area is related to the external diameter of the valve. Supra-annular implantation (where the sewing cushion lies above the aortic annulus) may be helpful, but the outer diameter of the valve body is a controlling factor. Many aortic prosthetic valves manufactures place emphasis on the placement of the prosthesis (sub-annular, intra-annular and supra-annular) in order to draw attention to the importance of implanting a prosthesis with the largest possible valve orifice diameter. Supra-annular placement is often preferred because usually a valve with a larger internal orifice diameter can be implanted. However, in patients with small aortic roots, even the most advantageous super-annularly mounted prosthesis may still result in a clinically significant aortic stenosis.

To address this problem some surgeons enlarge the aortic root by implanting a surgical patch of pericardium into a segment of the aortic root, hence allowing the implantation of a larger than otherwise aortic prosthesis. This is a complex and technically difficult operation that significantly increases cardiopulmonary bypass time, and carries an increased risk of hemorrhage.

Pediatric patients may also be afflicted with congenital aortic valve disease. In addition to aortic stenosis, pediatric patients may have subaortic stenosis (a constriction or narrowing below the aortic annulus. This, like aortic stenosis restricts the flow of blood passing into the aorta. This condition may be congenital or may be due to a particular form of cardiomyopathy known as “idiopathic hypertrophic subaortic stenosis”. The particular problem with pediatric patients is that normal growth will slowly outstrip the replacement aortic valve, hence the patient has to have repeat operations as growth takes place if cardiac damage is to be avoided.

### BRIEF DESCRIPTION OF THE DRAWINGS

FIG. 1 is a side elevation of one embodiment of the aortic root expansion apparatus in the unexpanded condition with the detachable irreversible dilation member in place.

FIG. 2 is an end view of FIG. 1 in the direction of arrow A in FIG. 1

FIG. 3 is an end view of FIG. 1 in the direction of arrow B in FIG. 1

FIG. 4 is side elevation of the preferred embodiment of the aortic root expansion apparatus with the irreversible dilation member absent.

FIG. 5 is an end view of FIG. 4 in the direction of arrow D in FIG. 4

FIG. 6 is an exploded isometric view of the aortic root expansion apparatus of FIG. 1 with the irreversible dilation member absent.

FIG. 7 is a cross-section view of the aortic root expansion apparatus taken along line CC of FIG. 2.

FIG. 8 is a cross-section view of the aortic root expansion apparatus taken along line EE of FIG. 7.

FIG. 9 is a partial cross-section view of the aortic root expansion apparatus taken along line CC of FIG. 2 but in the expanded condition.

FIG. 10 is an end view taken in the direction of arrow F in FIG. 9.

FIG. 11 is a cross-section view taken along line GG of FIG. 9

FIG. 12 is a plan view of one embodiment of the irreversible dilation member in its pre-coiled flat form

FIG. 13 is a plan view of the an alternative embodiment of the irreversible dilation member in its pre-coiled flat form

FIG. 14 is a side view of the irreversible dilation member shown in FIG. 12 in the flat, pre-coiled form

FIG. 15 is an enlarged cross-section taken along line HH of FIG. 12.

FIG. 16 is a side elevation of the irreversible dilation member in its unexpanded form.

FIG. 17 is an end view of the irreversible dilation member in its unexpanded form taken in the direction of arrow I of FIG. 16.

FIG. 18 is an enlarged part end view of the irreversible dilation member in its unexpanded form taken in the direction of arrow I of FIG. 16.

FIG. 19 is an end elevation of irreversible dilation member in an expanded form taken in the direction of arrow I of FIG. 16.

FIG. 20 is a side elevation of an alternative embodiment of the aortic root expansion apparatus in the unexpanded condition with an elastic sheath member in place.

FIG. 21 is an end view taken in the direction of arrow J in FIG. 20.

FIG. 22 is a part cross-section side elevation taken along line KK of FIG. 21 in the unexpanded condition with an elastic sheath member in place.

FIG. 23 is a part cross-section side elevation taken along line KK of FIG. 20 in the expanded condition with an elastic sheath member in place.

### SUMMARY OF THE INVENTION

The present invention includes an apparatus and method for stretching and expanding the aortic root, and maintaining the dilation while a replacement valve is implanted. The normal aorta is elastic. In patients with small aortic roots the apparatus and method of this invention is most useful as it allows the surgeon to controllably dilate the aortic root using a thin walled spiral cylinder that is irreversibly dilated by an expansion apparatus. Following removal of the reversible expansive apparatus the irreversible dilation member temporarily remains in the expanded aortic root during an aortic valve prosthesis placement within the dilation member at the level of the aortic annulus. Implantation sutures, previously placed, are tied following the removal of the dilation member.

Aortic valve prostheses include: mechanical valve prostheses; biological prostheses (or xenograft prostheses); autograft prostheses (homograft prostheses); synthetic leaflet valve prostheses; patient's tissue constructed valves; patient's tissue grown valves; or any other type or kind of valve prostheses, including endoscopically or minimally invasively implanted valve prostheses.

In one embodiment of the invention the expansion apparatus is a mechanical mechanism, but in an alternative embodiment the expansion apparatus may be an inflatable balloon. Alternative embodiments of the irreversible dilation member are an aortic root stent that will remain implanted in the aortic root and a sub aortic stent expanded and implanted just below the aortic valve orifice.

The embodiments disclosed herein allows an adult patient with a small aortic root to have a larger aortic valve implanted. The embodiments eliminate the added operative time and potential complications associated with surgical aortic root enlargement, by permitting the surgeon to implant a larger prosthesis into the aortic root, especially a small aortic root. In pediatric patients expansion of the aortic root using the method and apparatus allows a significantly larger valve to be implanted, thereby reducing the number of re-operations the patient must endure. In pediatric sub-aortic stenosis the embodiment may be used to implant a sub-aortic stent immediately below the normal or replacement valve prosthesis, to eliminate an obstruction.

### DETAILED DESCRIPTION OF THE INVENTION

The present disclosure includes several alternative expansive devices and methods to allow the implantation of a larger than otherwise aortic valve prosthesis in the aortic annulus of a patient. Although aortic valve replacement is illustrated, those skilled in the art will understand that the devices and methods are also applicable for use in any of the three other valves of the heart, (i.e. the mitral, pulmonary and tricuspid valves), or other orifices in the human body. In this description like numbers will be used to identify like elements according to the different figures which illustrate the invention. However, it should be noted that the specific constructional details, including materials, shapes and number of certain elements, may be varied from those shown.

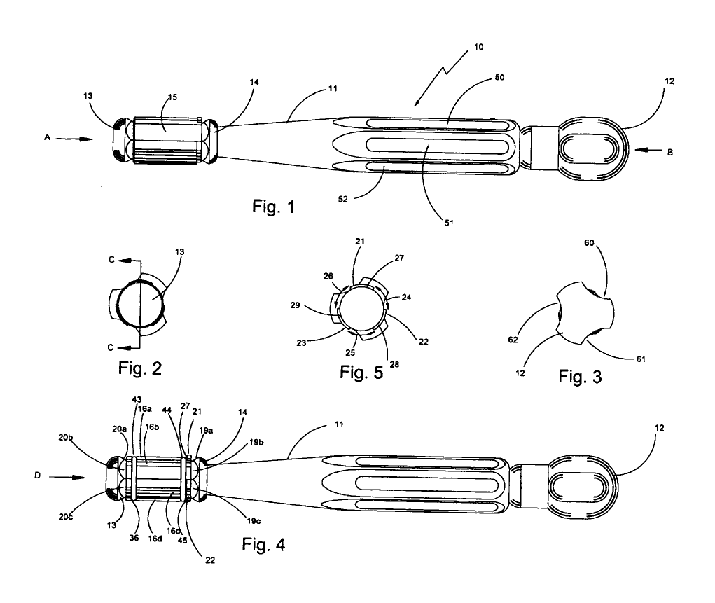

One embodiment of an expansion device 10 is illustrated in FIG. 1 which is a side elevation of the expansion device 10 in its normal (contracted) state. Shown are a handle member 11, an actuating knob 12, distal actuating member 13, and proximal actuating member 14, and an irreversible dilation member 15. FIG. 2 shows an end elevation taken in the direction of arrow A in FIG. 1, and FIG. 3 shows an end elevation taken in the direction of arrow B in FIG. 1.

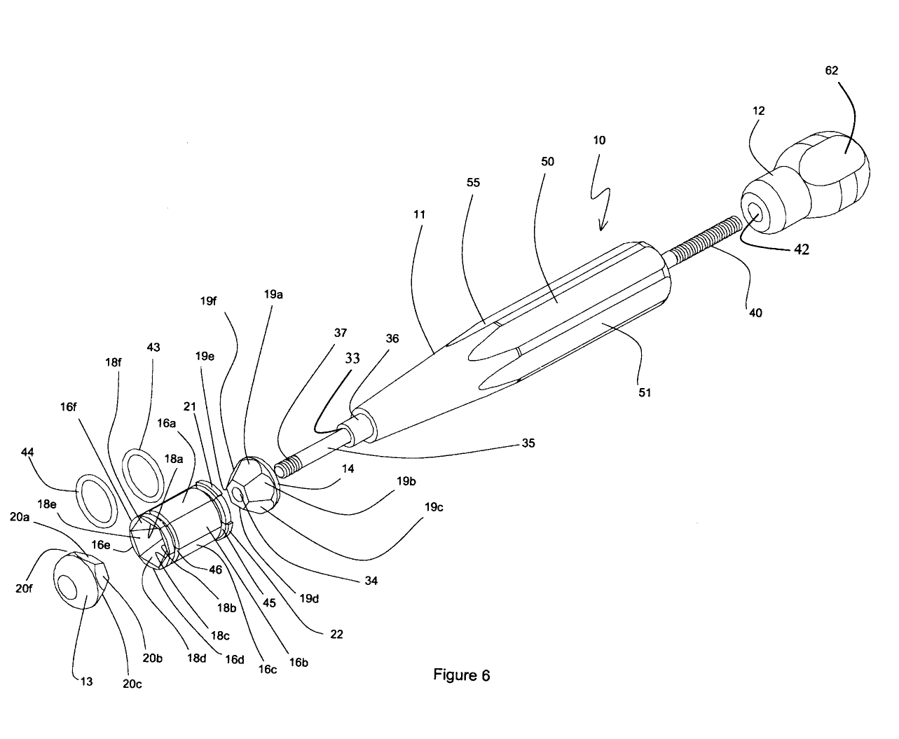

FIG. 4 is a side elevation of the aortic root expansion apparatus 10 with the irreversible dilation member 15 removed for clarity of illustration. FIG. 6 is an exploded isometric view of the aortic root expansion apparatus 10 with the irreversible dilation member absent. Six expansion members (shown best in FIG. 11) 16a, 16b, 16c, 16d, 16e, and 16f each have an angular plane face at either end. Faces 17a, 17b, 17c, 17e, 17f, and 17g are situated at the proximal ends, and faces 18a, 18b, 18c, 18d, and 18g are situated at the distal end. These planar surfaces contact corresponding angled plane face 19a, 19b, 19c, 19d, 19e, 19f and 19g on actuating member 14 and angled plane face 20a, 20b, 20c, 20d, 20e, 20f and 20g on dilating actuating member 13 (only faces 20a, 20b and 20c are shown) respectively. Three of the expansion members 16a, 16c and 16e have flanges 21, 22, and 23 respectively creating spaces 24, 25 and 26 (seen best in FIG. 5 through which the implanting sutures may pass). Gap 24 lies between flanges 21 and 22, gap 25 lies between flanges 22 and 23, and gap 26 lies between flanges 23 and 21. Flange 21 has distal face 27, flange 22 has distal face 28, and flange 23 has distal face 29. These faces prevent movement of irreversible dilation member 15 in the proximal direction.

Rod member 35 passes through clearance hole 33 in handle 11, through clearance hole 34 in actuating member 14 and passes within the inner faces 32a, 32b, 32c, 32d, 32e and 32f of expansive members 16a, 16b, 16c, 16d, 16e and 16f respectively, as shown in FIG. 11. Handle member 11 has a snout 36 at its distal end that is an interference press fit into recess 39 in expansive member 14. However, those skilled in the art will realize the expansive member 14 could equally well be an integral part of handle 11.

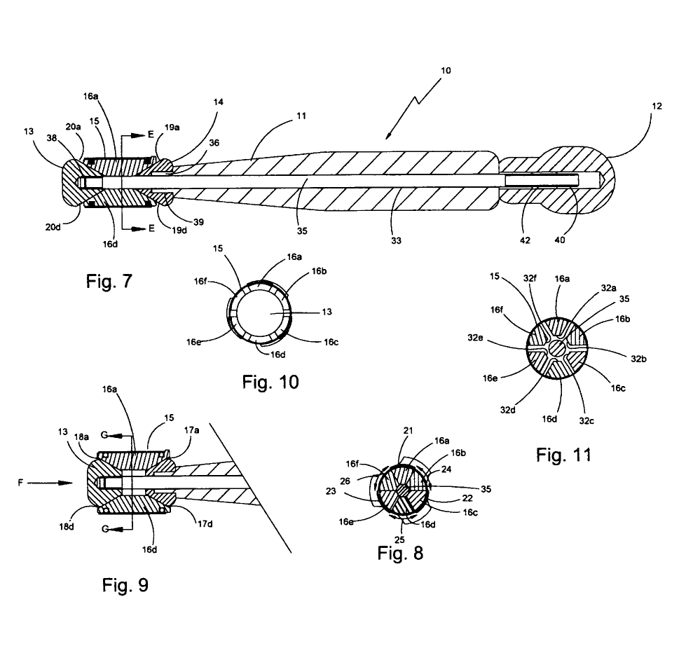

Distal end of rod member 35 is terminated with a threaded male portion 37 that engages in a corresponding female threaded portion 38 in actuating member 13 (see FIG. 7). The proximal end of rod member 35 is likewise terminated with a male threaded section 40 that rotatably engages with a corresponding female threaded recess 42 contained in actuating knob 12 (if knob member 12 is of aluminum or a plastic material threaded recess 42 may preferably be a stainless steel Helicoil™ type thread insert). The expansive members are 16a-16f and are inwardly restrained by proximal elastic members 43, 44 contained within annular groves 45, 46. The proximal elastic member 43 and distal elastic member 44 are preferably “O” rings of an elastomeric material with suitable elongation or stretch and durometer. In an alternative embodiment of the invention the inwardly acting members may be tension springs preferably of a corrosion resistant and/or biological compatible material such as stainless steel with suitably joined ends such as intertwined loops.

Handle 11 has several optional axial recesses 50, 51, 52, 53, 54 and 55 to provide enhanced hand grip, (only 50, 51, 52 and 55 are shown in FIG. 1 and FIG. 6. Likewise knob 12 has optional recesses 60, 61, 62 to provide enhanced grip, especially when the surgeon's gloves are wet.

Referring now to FIG. 7 there is shown a cross-section view taken along line CC of FIG. 2. This shows expansion device 10 in its initial (unexpanded) condition with the irreversible dilation member 15 (also in its initial condition) in place. Clockwise rotation of knob 12 relative to handle 11 causes distal element 13 to be drawn towards proximal element 14, thus causing expansive elements 16a-f to slide up inclined planes 19a-f and 20a-f and hence move radially outwardly, in turn causing and irreversible dilation member 15 to expand. FIG. 8 shows cross-section view taken along line EE of FIG. 7. This shows the preferred embodiment of the expansion device 10 in its initial (unexpanded) condition with the irreversible dilation member 15 (also in its initial, unexpanded initial condition) in place. FIG. 9 shows a partial cross-section view of the distal expansive end of expansion device 10 taken along line CC of FIG. 2, but in an expanded condition. Knob 12 has been tightened in a clockwise direction (the screw threads are preferably right-handed threads), thus drawing distal expansive member 13 axially towards proximal expansive member 14, and hence causing members 16a-f to move radially outwardly. Consequentially member 15 is irreversible dilated. FIG. 10 shows an end view taken in the direction of arrow F of FIG. 9. FIG. 11 shows a cross-section view taken along line GG of FIG. 9. In FIG. 11 six expansion members 16a, 16b, 16c, 16d, 16e, and 16f are shown, but those skilled in the art will realize that the number of expansive members is unimportant to the invention, as two or three to twenty or more expansive elements could be used with similar effect.

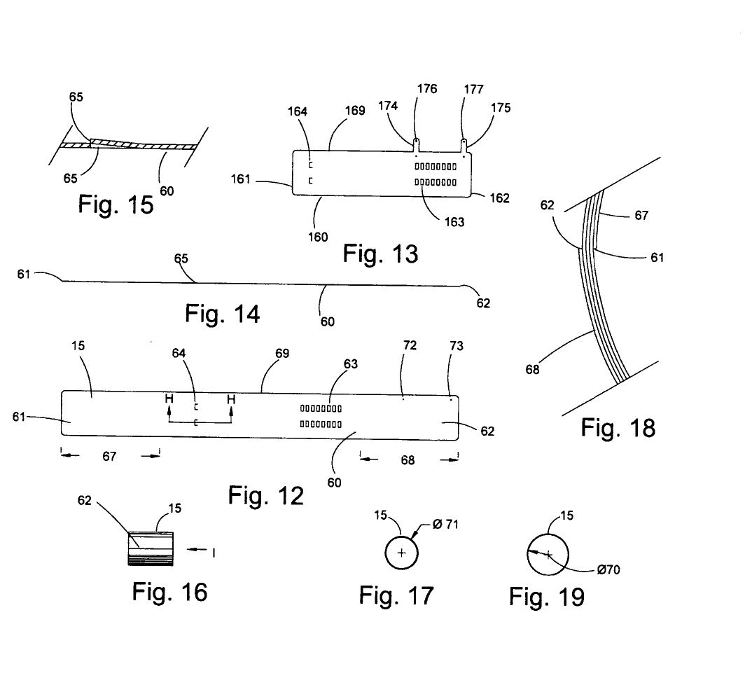

FIG. 12 shows the irreversible dilation member 15 in its linear (pre-curved) form 60. Strip 60 has a left-hand end 61 and a right-hand end 62. In the preferred embodiment strip 60 has an array of rectangular fenestrations 63 and at least one column of dimensionally appropriate and corresponding partial rectangular cuts 64 that are deformed into catches 65, best shown in FIG. 15 which is an enlarged cross sectional view taken along line HH of FIG. 12. Holes 72, 73 are located near the proximal edge 69 of strip 60, hole 73 being close to the right-hand edge 62 of strip 60. Strip 60 is rolled, coiled or otherwise formed spirally into irreversible dilation member 15, as shown in side elevation in FIG. 16 and end elevation in FIG. 17 and in enlarged part end view FIG. 18.

When the expansive apparatus is actuated irreversible dilation member 15 is expanded from its initial diameter to a predetermined larger diameter shown in FIG. 19, the column of catches 65 engage in a corresponding column of fenestrations 63, thus preventing irreversible dilation member 15 from returning towards its initial diameter. The shape, size and arrangement of the fenestrations and catches are not important to the retentative action, and that many variations are possible. After the strip is rolled or coiled into a spiral cylinder forming dilation member 15, a length of suture may be tied to each of the two holes 72, 73. Irreversible dilation member 15 is placed on the expansion apparatus such that edge 69 lies against wall faces 27, 28, and 29 shown in FIG. 5 and the sutures are and brought past the handle section. Following dilation, the expansion apparatus is removed, and the valve is inserted into the annulus. Thereafter the two sutures attached to holes 72, 73 may be pulled to remove the irreversible dilation member 15 from the aorta leaving the valve in the aortic annulus. Alternatively, dilation member 15 could be grasped using artery forceps to pull the member from the aortic annulus.

In shaping strip 60 into a spiral cylindrical irreversible dilation member 15 it may be advantageous to form the left hand portion of the strip 60, designated as 67 (that will lie innermost of the coiled member) into an inner diameter that is larger than its fully expanded internal diameter (designated Ø70 in FIG. 19), and the right-hand end portion 68 of strip 60, into a diameter that is smaller than its initial unexpanded diameter Ø71 shown in FIG. 17. The former is desirable because when the device is expanded and the temporary expansive inner member is relaxed and removed, the inner diameter of the irreversible dilation member 15 will remain outwardly expanded and thus not interfere with the insertion of the valve prosthesis. The latter is desirable to prevent self expansion of irreversible dilation member 15. These features are important because the largest possible valve replacement should be inserted for implantation.

FIG. 13 shows an alternative embodiment of the irreversible dilation member 15 in its linear (pre-curved) form 160. Strip 160 has a left-hand end 161 and a right-hand end 162. In the alternative embodiment strip 160 has an array of rectangular fenestration 163 and at least one column of corresponding partial rectangular cuts 164 that are deformed into catches similar to catch 65 shown in FIG. 15. Strip 160 is formed into spiral cylinder, similar to that shown in FIG. 17 and in enlarged part end view FIG. 18, but having fewer spirals than is produce by longer strip 60. This reduction has the advantage of diminished overall spiral cylinder wall thickness, but has the disadvantage that the outermost and innermost surfaces of member 15 are not perfectly smooth. For example, the outer wall surface has the array of fenestrations (163) and the innermost wall has cuts similar to that indicated as 65 in FIG. 15. In yet another embodiment two lugs 176 and 177 protrude from the right-hand portion of the strip 161, lug 175 being close to strip end 162. The spacing between the lugs and the width of the lugs is such that when the strip is coiled or rolled into a spiral cylinder and placed onto the expansive apparatus such that edge 169 abuts wall faces 27, 28, and 29 (shown in FIG. 5), the lugs 176 and 177 lie in gaps 24 and 24, or 25 and 26 independent of whether the cylinder is contracted or expanded. Lug 176 has a hole 176 near its proximal end, and lug 175 likewise has a hole 177 near its proximal end. A length of suture may be tied to each of these two holes, and brought past the handle section. These sutures may later be pulled to remove the irreversible dilation member 15 from the aortic annulus. Alternatively the lugs could be grasped using artery forceps to pull the irreversible dilation member 15 from the aortic annulus.

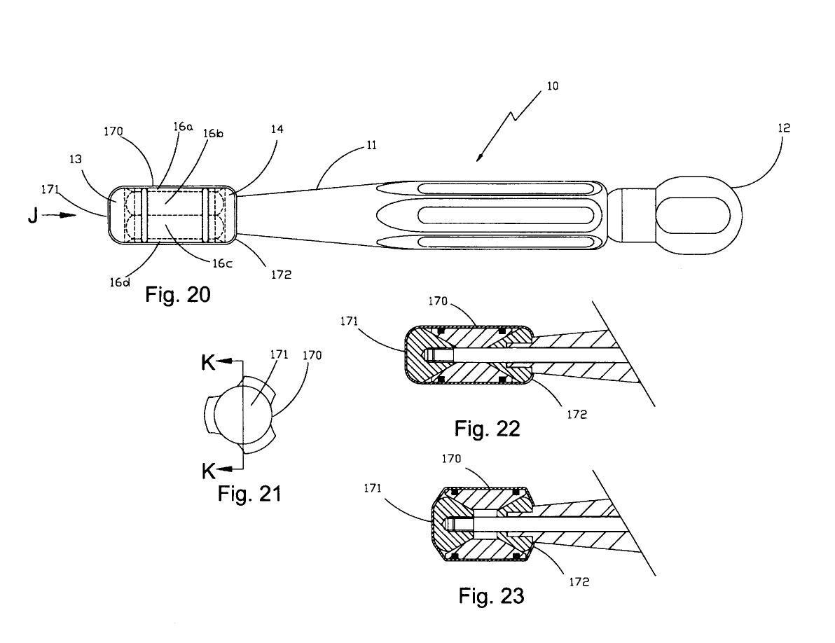

A further alternative embodiment of the invention is shown in FIG. 20. This is a side elevation of an aortic root expansion apparatus 10 in the unexpanded condition with an elastic sheath member 80 stretched over distal actuating member 13, expansive elements 16a-f and proximal actuating member 14 (expansive elements 16a, 16b, 16c and 16d are shown as phantom lines). Elastic sheath 170 is an open ended cylindrical form with closed end 171 and open end 172, molded to match the contours of distal and proximal actuating members 13, 14 respectively. Elastic sheath 170 may be molded using silicon elastomer or a biocompatible rubber such as Santoprene thermoplastic rubber type 281-55, however silicon elastomer might be better suited because of its superior elongation characteristics.

FIG. 21 is and end elevation in the direction of arrow J of FIG. 20, and FIG. 22 shows a part cross-section side elevation taken along line KK of FIG. 21. FIG. 23 shows a part cross-section side elevation taken along line KK of FIG. 21 of an alternative embodiment of the aortic root expansion apparatus in the expanded condition with an elastic sheath member 170 in place. This alternative embodiment may be useful for deployment in the calcified aortic root, or when it is desirable to place a permanent aortic annulus stent in conjunction with the use of a tissue valve prosthesis.

### EXAMPLE

### Structure

A prototype embodiment had an outer diameter of the assembled expansive member in its initial condition of 17.7 mm and the outer diameter measured across the midpoints of the expansive member in its expanded state of 22.9 mm. The irreversible dilation member 15 was made from laser cut stainless steel 0.006″ thick, using a flat pattern similar to that shown in FIG. 13 (but shorter and with two fewer columns of fenestrations, and without the two lugs 174, 175 and corresponding holes 174, 175). The strip was rolled into a spiral cylinder using a small, purpose built roll bender. A dead pig heart was procured, the aorta opened and the aortic annulus size measured using an ATS Medical, Inc. aortic valve sizer. A 19 mm sizer was found to be a smug fit in the aortic annulus, but it could be pushed though the annulus with slight force. A 21 mm sizer would not go into the aortic annulus, but could be forced through using high force. The sizer was pulled out and the 19 mm sizer tried again, showing that the aortic root had not been stretched by forcing the 21 mm sizer through the annulus. The prototype apparatus of this invention was then inserted into the aorta so that the irreversible dilation member 15 transversed the aortic root. Following actuation to the last fenestration in the dilation member 15, followed by de-activation and removal of the expansion apparatus, the inner lumen of dilation member 15 was again measured. Whereas, a 22 mm sizer was very loose in the dilation member 15, a 23 mm sizer would not quite go in. It was estimated that the lumen size was 22.8 mm. Therefore two extra rows of fenestrations were added to the flat pattern design, resulting in the flat pattern shown in FIG. 13. A modified prototype should be capable of expanding the aortic root from 19 mm to 23 mm, or more. Clearly, the device could be manufactured in a wide variety of sizes, smaller for pediatric patients, and larger for large adult patients.

Handle member 11 may be of aluminum (preferably anodized), titanium, stainless steel or other metal, or alternatively of a wide variety of plastic material such as polyethylene, ABS or acetyl copolymer. Likewise expansive member 14 may be of a stainless steel, suitable plastic such as acetyl copolymer, or ABS or other plastic, or metal such as stainless steel, aluminum or titanium, or other metals. Irreversible dilation member 15 may be made of a variety of materials. For example although stainless steel or titanium may be desirable, aluminum (preferably anodized or plastic coated) could be used. Alternatively, this irreversible dilation member could be of a plastic material such as a polyester film that is heat set into the correct shape. The thickness of strip 60 used to form irreversible dilation member 15 depends upon the material used, but generally will lie in the range 0.002″ to 0.010″ thick. The instrument could be supplied as a disposable device, or could be supplied as a reusable instrument. The disposable instrument would not require cleaning before use.

### Method of Use

The example given here is for aortic valve replacement with a rigid mechanical aortic valve prosthesis, but the method is equally applicable to mitral, tricuspid and pulmonary valve replacement, especially in pediatric patients who will grow in size and where a larger than otherwise prosthesis is especially important as it may reduce the number of subsequent cardiac surgical operations.

Following excision of the diseased aortic valve, the surgeon measures the internal annulus diameter. This measurement is usually made by using an appropriate valve “sizer”. The sizer is usually a cylindrical template generally unique to individual valve replacement manufacturer. The configuration, form and dimensions vary with prosthetic, or bioprosthetic, valve type, models and especially valve size.

Aortic root size estimation may also be made radiographically, or using ultrasound visualization, or by using the chosen prosthetic heart valve manufacturer's valve sizers. Should the aortic root annulus be found to be unusually small, as compared to the average patient of the similar body surface area, the surgeon may elect to attempt to stretch and enlarge the aortic root using the method and apparatus described herein so be able to implant a larger than otherwise prosthetic valve into the valve annulus.

The sequence of the valve implantation would be expected to approximately follow the sequence listed below:
a. Excise the diseased valve
b. Decalcify the valve annulus, as far as possible, if necessary.
c. Place multiple prosthetic valve implantation sutures (usually with pledgets) into and around and the valve annulus.
d. Pass the implantation sutures with needles attached through the lumen of the irreversible dilation member 15.
e. Place sutures or suture needles in one or more suture organizers, suture retainers (or magnetic retainers for holding and retaining the suture needles).
f. Slide the irreversible dilation member 15 with the implanting sutures over the distal and proximal actuating members 13, 14.
g. Introduce the assembly (the expansive device with the implanting sutures mounted on the aortic root expansive member) such as to straddle the aortic annulus.
h. Actuate the expansion device 10, thus increasing the diameter of the irreversible dilation member 15. As the dilating device is expanded a series of click may be heard as catches 65 engage in successive fenestrations 63 in 15. Continue actuation until the desired diameter is obtained.
i. De-actuate the expansive device 10, thus reducing its outer diameter until it is a loose fit in the now fixed irreversible dilation member 15. The latter remains in its expanded state because catches 60 have engaged fenestrations 63, thus preventing a return of 15 to its lesser diameter.
j. Remove the expansive device 10, leaving the irreversible dilation member 15 and the implantation suture in place across the annulus.
k. Measure the distance of the distal edge part of the irreversible dilation member 15 to the aortic annulus using a simple scalar fixture.
l. Use a manufacturer's valve sizer to determine the size of appropriate valve that will just slide into the internal diameter of the dilating device. Because the implantation sutures lie within the irreversible dilation member 15, an appropriate allowance should be made for the additional space taken up by these sutures, that on implantation will pass through, not over the sewing cushion of the valve prosthesis.
m. Select the appropriate size and type of valve that would just slide into the irreversible dilation member 15 if the implantation sutures were absent.
n. Place the needles of the implantation sutures through the valve sewing cushion as in routine valve surgery.
o. Pull the implantation sutures through the sewing cushion and cut-off the needles, as is current surgical practice.
p. Insert the valve into the irreversible dilation member 15.
q. Push the valve down the lumen of the irreversible dilation member 15 until the sewing cushion lies opposite the valve orifice. This should have may have been measured in step (k).
r. Holding the valve in position relative to the annulus (using the valve holder supplied with the valve), carefully remove irreversible dilation member 15 either using the sutures tied to it, or be grasping a protruding lug using artery forceps, and gently pulling axially away from the annulus.
s. As the irreversible dilation member 15 is withdrawn from the annulus, the expanded aortic annulus will contract over the prosthetic valve sewing cushion.
t. Retaining the position of the valve, relative to the annulus, the implantation sutures are tightened. They are then tied and cut close to the sewing cushion so that the suture tails cannot impinge on the valve orifice (in the case of mechanical valves).
u. Close the aorta and complete the operation.

Immediately following the removal of the irreversible dilation member 15 from the aortic annulus the annulus will elastically attempt to return to its former or near former diameter. When a mechanical prosthetic is implanted this “recoil” should not present any difficulties because most mechanical valve prostheses are manufactured from pyrolytic carbon or metal with high compressive strength. However, tissue valves, even those mounted in stents or frameworks are flimsy by comparison. The contracting force of the aortic annulus could easily seriously distort a porcine or pericardial bioprostheses. Worse, if a stentless aortic bioprostheses such as the Medtronic Freestyle™ valve or an autograft vale is implanted they would be seriously distorted and the advantage of aortic annulus dilation would be lost. In such cases, or where the patient has sub aortic stenosis, an aortic annulus stent or a sub aortic annulus stent could be inserted in or just below the aortic annulus. The unexpanded stent is placed on the expansive device 10, introduced into or below the aortic annulus, and expanded. The expansion apparatus is then returned to its initial diameter and removed, leaving the stent in the aortic annulus or in the sub aortic position respectively.

The amount the aortic annulus is expanded will be controlled by and known to the surgeon. For example:
a) The diametral size increase may be known by counting the number of complete rotations handle 12 is turned.
b) By counting the “clicks” as catches 65 spring into fenestrations 63
c) By incorporating a simple linear scale into the apparatus that is calibrated in diametral increase.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

1. A cardiac valve annulus expansion device comprising:
an expansion member defining an expansion circumference corresponding to an inner circumference of a cardiac valve annulus; and
an actuation member operatively associated with the expansion member wherein actuation of the actuation member causes radial expansion of the expansion circumference.

2. The cardiac valve annulus expansion device of claim 1 wherein actuation of the actuation member selectively causes radial contraction of the expansion circumference.

3. The cardiac valve annulus expansion device of claim 2 further comprising a dilation member operatively associated with the expansion member, the dilation member being expandable from a first diameter corresponding to an inner circumference of a cardiac valve to a second larger diameter.

4. The cardiac valve annulus expansion device of claim 3 wherein expansion of the expansion circumference causes expansion of the dilation circumference.

5. The cardiac valve annulus expansion device of claim 4 further comprising means associated with the dilation member preventing contraction of the dilation circumference.

6. The cardiac valve annulus expansion device of claim 1 wherein the expansion member comprises a mechanical device.

7. The cardiac valve annulus expansion device of claim 1 wherein the expansion member comprises an inflatable balloon.

8. A cardiac valve expansion device comprising:
an irreversible dilation member, the irreversible dilation member comprising a band formed into a cylinder of a first diameter and means operatively associated with the band for irreversibly maintaining the band in a second diameter greater than the first diameter;
an expansion member defining an expansion circumference receiving an inner diameter of the irreversible dilation member, the expansion circumference corresponding to an inner circumference of a cardiac valve annulus; and
an actuator operatively associated with the expansion member to expand the expansion circumference of the irreversible dilation member.

9. The cardiac valve expansion device of claim 8 further comprising the actuator being configured to cause radial contraction of the expansion circumference.

10. The cardiac valve expansion device of claim 8 wherein the second diameter of the irreversible dilation member is selectable from a plurality of diameters greater than the first diamater

11. A method of expanding a cardiac valve annulus comprising:
providing a cardiac valve annulus expansion device including an expansion member adjacent to a cardiac valve annulus; and
expanding the expansion member.

12. The method of expanding a cardiac valve annulus of claim 11 further comprising expanding a dilation member in contact with the cardiac valve annulus.

13. The method of expanding a cardiac valve annulus of claim 12 further comprising:
contracting the expansion member; and
removing the cardiac valve expansion device from the vicinity of the cardiac valve annulus.

14. A method of cardiac valve replacement surgery comprising:
excising a cardiac valve;
providing a dilation member in contact with an inner diameter of the cardiac valve annulus;
expanding the annular circumference of the dilation member; and
replacing the cardiac valve.

15. The method of claim 14 further comprising the dilation member being irreversible and irreversibly expanding the dilation member.

16. The method of claim 15 further comprising removing the dilation member.

17. The method of claim 15 further comprising leaving the dilation member in the valve annulus to function as a stent.

18. The method of claim 15 further comprising providing an irreversible dilation member in a sub-aortic position, expanding the annular circumference of the dilation member and leaving the dilation member in the sub-aortic position to function as a stent.
